(19) United States
(12) Patent Application Publication
(10) Pub. No.: US 2002/0002202 A1
(43) Pub. Date: Jan. 3, 2002

(54) Use of fumarate salt of L-carnitine or its alkanoyl derivatives in ischaemia

(76) Inventors: Claudio Cavazza, Rome (IT); Arduino Arduini, Rome (IT)

Correspondence Address: NIXON & VANDERHYE P.C., 8th Floor, 1100 North Glebe Rd., Arlington, VA, 22201-4714, US

(21) Appl. No.: 09/858,639

(22) Filed: May 17, 2001

Related U.S. Application Data
(63) Continuation of application No. PCT/IT99/00369, filed on Nov. 16, 1999.

(30) Foreign Application Priority Data
Nov. 26, 1998 (IT) RM98A000726
May 25, 1999 (IT) RM99A000328

(51) Int. Cl. A61K 31/195; A61K 31/221

(52) U.S. Cl. 514/546; 514/561

(57) ABSTRACT

The invention described herein relates to the use of L-carnitine or one of its alkanoyl derivatives with formula (I) where R is an alkanoyl group with from 2 to 8 carbon atoms, and X⁻ is the acid fumarate anion, for the preparation of a medicine useful in the treatment and prevention of organ ischemia.

The invention described herein relates to the use of L-carnitine acid fumarate and its alkanoyl derivatives (hereinafter referred to simply as L-carnitine fumarate or alkanoyl L-carnitine fumarate) to prepare a composition suitable for reducing, in a broad range of users and/or patients, the risk of onset of organ ischemia, and for preventing and/or therapeutically treating it, particularly as affecting the cardiocirculatory apparatus.

Accordingly, the composition may take the form and exert the activity of a food supplement or of an actual medicine in its own right, depending upon whether the action which the composition is intended to exert is one of support or prevention or is meant to be strictly therapeutic according to the particular subjects it is to be used for.

### BACKGROUND TO THE INVENTION

Organ ischemia is caused by an imbalance between the oxygen requirement on the part of the tissue and the availability of oxygen from the bloodstream. In the particular case of cardiac ischemia, this manifests itself in the form of a typical set of symptoms, known as angina pectoris. The causes are multiple and among them one should mention a reduced ability on the part of coronary circulatory system to supply oxygen, for instance as a result of the presence of atheromatous plaques. One possible consequence of ischemia is myocardial infarction.

Myocardial ischemia can also be silent and detectable only by means of clinico-instrumental investigations.

The therapy currently available is based mainly on the administration of coronary vasodilator drugs, which, owing to the specific demands of treatment of the symptoms, must be as fast-acting as possible. Calcium antagonists, β-adrenergic antagonists and antiplatelet agents should also be mentioned.

Among the drugs which are still most commonly used today, we should mention the organic nitrates which, by releasing NO at the action site, exert a local vasodilatory action.

Amyl nitrite is used by inhalation in cases of angina attack, and nitroglycerin and organic nitrates of higher molecular weight are also used for preventing the attacks. Nitroderivatives are associated with a series of major side effects. The most common of these is headache, which may also be of substantial intensity. More serious is the fact that these drugs give rise to tolerance and their discontinuation causes a rebound effect. Nitroglycerin is also administered via transdermal release systems which, however sophisticated they may be, present problems of their own, such as permanence in the application site, controlled delivery of the drug, and patient compliance.

Calcium antagonists present the problem of excessive vasodilatation, with dizziness, hypotension, headache and nausea as a result, and establishing the correct therapeutic regimen is by no means an easy matter.

β-antagonists have consequences in terms of cardiac haemodynamics.

For a more extensive discussion of these aspects, the skilled reader is referred to Goodman & Gilman, The Pharmacological Basis of Therapeutics—9th edition, chapter 32.

It is well known that fumarate has a favourable action on the immature heart (Pearl, J. M. et al. Ann. Thorac. Surg., 57, 1636-1641; 1993).

It is also well known that acetyl L-carnitine improves glucose oxidation and prevents the accumulation of lactate in concomitant acidosis (Lopaschuk, G in Carnitine Today—C. De Simone and G. Famularo ed. Lands Bioscience 1997).

Salts of L-carnitine acid fumarate and its alkanoyl derivatives are described in patent EP 0 150 688. These salts, together with a large series of salts with other anions, selected from acid aspartate, acid citrate, acid phosphate, acid lactate, acid maleate, acid oxalate, acid sulphate and orotate, present the advantageous property of being non-hygroscopic, thus solving the problem, known to experts in pharmaceutical technology, of the substantial hygroscopicity of L-carnitine and its alkanoyl derivatives.

Alkanoyl derivatives of L-carnitine are known for their various uses in human or animal therapy.

The fumarates of these derivatives are described, for example, in EP 0 376 899, for the treatment of peripheral neuropathies, and, in EP 0 516 594, for the treatment of myopathies and neuronal degeneration and for the inhibition of proteolysis.

L-carnitine fumarate is a known, highly stable, non-hygroscopic compound. Its preparation and physicochemical properties are described, in fact, in U.S. Pat. No. 4,602,039, which is incorporated in this description for reference purposes.

The usefulness and commercial success of this salt of L-carnitine are due particularly to its lack of hygroscopicity. L-carnitine fumarate thus lends itself favourably to the preparation of solid dietetic, nutritional or pharmaceutical compositions that can be prepared using traditional-type mixing devices, tabletting machines and the like. Neither L-carnitine fumarate as raw material nor the finished products obtained from it present problems of processing, packaging and storage even over lengthy time periods and in unfavourable environmental conditions, i.e. in the presence of high relative humidity values (approximately 70%).

On the other hand, it is known that the high hygroscopicity of L-carnitine inner salt and its alkanoyl derivatives (acetyl L-carnitine, propionyl L-carnitine, etc.) poses complex problems of processing, stability and storage both for the raw materials and for the related finished products.

These problems have given rise to substantial efforts to synthesise non-hygroscopic salts of such carnitines which are solid and stable, even in prolonged storage conditions, and which can therefore be easily processed and formulated with the usual excipients, using traditional-type devices and which, in addition, pose no packaging problems when transformed into finished products.

The need to solve the problem of the hygroscopicity of L-carnitine and its alkanoyl derivatives has substantially increased since the use of these active ingredients was extended from the strictly ethical field and from the preparation of pharmaceutical compositions to the nutritional/dietetic field and to the production of food supplements. It is, in fact, in this field that solid compositions containing L-carnitine, such as tablets and capsules, constitute the preferred form of administration, in that they make it particularly easy for the users to take the active ingredient, complying with optimal dosage regimens. Whereas it is clear that L-carnitine fumarate provides a very satisfactory solution to the above-mentioned problem of the hygroscopicity of L-carnitine inner salt, it is equally clear that the increasingly wide-scale adoption of this L-carnitine salt and its resulting commercial success have so far been related exclusively to considerations of pharmaceutical technology and not to considerations of therapeutic or nutritional activity.

In other words, as far as its therapeutic or nutritional activity are concerned, L-carnitine fumarate has so far been regarded as being no different from L-carnitine inner salt or from other pharmacologically acceptable salts of L-carnitine.

Only very recently, the Applicant (in patent application EP 98830383.0, filed on 25.06.98 and thus not yet made public on the date of filing of the present application) drew attention to the fact that the gastrointestinal disorders (increased volume and fluidity of faeces and frequency of bowel movements in relation to the usual trend) caused by taking L-carnitine inner salt or L-carnitine L-tartrate are eliminated by administering a composition containing L-carnitine fumarate in an amount equivalent to the amount of exogenous L-carnitine required.

The cardioprotective activity of both L-carnitine and acid fumarate is well known. For example, U.S. Pat. No. 4,649,159 (Fanelli/Sigma-Tau) describes the usefulness of L-carnitine in the therapeutic treatment of ischemia and of myocardial anoxia. U.S. Pat. No. 4,656,191 (Fanelli/Sigma-Tau) describes its usefulness in the therapeutic treatment of arrhythmias and of congestive heart failure.

The cardioprotective effect of acid fumarate has been verified in the perfused rat heart (La Plante et al. “Effects and metabolism of fumarate in the perfused rat heart. A ¹³C mass isotopomer study”, Am. J. Physiol. 272:E74-E82, 1997) and on the immature myocardium (Pearl J. M. et al. “Fumarate enriched blood cardioplegia results in complete functional recovery of immature myocardium”, Ann. Thorac. Surg. 57: 1636-41, 1993).

### SUMMARY OF THE INVENTION

It has now surprisingly been found that the use of acid fumarate of L-carnitine or of one of its alkanoyl derivatives exerts a protective and thus a therapeutic or preventive activity on ischemic organs, and particularly on the ischemic heart.

The subject of the invention described herein is therefore the use of a compound with formula (I):

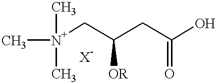

where R is an alkanoyl group with from 2 to 8 carbon atoms and X⁻ denotes the anion of acid fumarate, for the preparation of a medicine useful in the prevention and/or therapeutic treatment of cardiac ischemia.

One of the fundamental advantages of the invention described herein consists in the fact that treatment with fumarate of L-carnitine or one of its alkanoyl derivatives is practically devoid of major side effects.

In addition, also by no means negligible is the fact that L-carnitine or alkanoyl L-carnitine fumarate is a non-hygroscopic salt and is thus easily manageable in the preparation of medicines.

The invention described herein is based on the discovery that L-carnitine or alkanoyl L-carnitine and jointly exert a potent synergistic effect as compared to the known cardioprotective action exerted by the individual components.

Though this unexpected synergistic effect also occurs in the presence of a mixture of L-carnitine or alkanoyl L-carnitine inner salt or another of its pharmacologically acceptable salts and fumaric acid (in which the two components are present in substantially equimolar amounts) or also as a result of co-administration (i.e. simultaneous administration) or sequential administration of L-carnitine or alkanoyl L-carnitine inner salt or one of its pharmacologically acceptable salts and fumaric acid, it is clear, on the basis of the pharmaceutical technology considerations outlined above, that the use of L-carnitine or alkanoyl L-carnitine fumarate is substantially preferred.

The subject of the invention described herein is therefore the use of L-carnitine or alkanoyl L-carnitine acid fumarate for the preparation of a composition suitable for reducing the onset of organ ischemia, and for preventing and/or therapeutically treating it, particularly as affecting the cardiocirculatory apparatus in a broad range of users or patients.

### BRIEF DESCRIPTION OF THE FIGURES

FIG. 1 illustrates the treatment schedule, where the letters A-F denote the heart effluent sampling times for the measurement of metabolites.

FIG. 2 shows the effect of carnitine (A) and carnitine fumarate (B) on creatine phosphate and ATP.

FIG. 3 compares lactate (A) with succinate (B) released by the heart, as measured in the effluent.

FIG. 4 illustrates the release of malate.

FIG. 5 illustrates the release of LDH.

FIG. 6 illustrates the production of lactate.

### DETAILED DESCRIPTION OF THE INVENTION

In the context of the invention described herein, what is meant by organ ischemia is a reduced oxygen supply to the tissue in relation to the metabolism requirement.

As examples of organ ischemia, cardiac ischemia, cerebral ischemia and renal ischemia are mentioned.

As mentioned previously, the fumarates of L-carnitine or alkanoyl L-carnitine are non-hygroscopic salts, and this characteristic proves advantageous in the preparation of compositions, and particularly medicines, as solid oral forms.

The composition according to the invention is prepared using entirely conventional techniques which are part and parcel of the experience of the person having ordinary skill in the art.

Advantageously, the composition according to the invention may take the form of an oral pharmaceutical composition, such as, for instance, capsules, tablets, powders, granules, or lyophilised compounds which can be reconstituted in drinkable liquid forms at the time of use.

Injectable forms, both intravenous and intramuscular, are also envisaged.

For general indications as to the preparation of pharmaceutical forms, the skilled reader is referred to “Remington's Pharmaceutical Sciences Handbook—Mack Pub.”

A controlled-release pharmaceutical form may also be advantageous.

The composition may be administered orally and take the form of a food supplement, or of a medicine which can be administered orally or parenterally.

Examples of suitable administration forms of the composition consist of tablets, pills, granules, syrups, ampoules or drops.

The composition may additionally contain at least one other active ingredient and/or pharmacologically acceptable excipient. This additional active ingredient is preferably selected from the group consisting of alkanoyl L-carnitines in which the alkanoyl has 2-6 carbon atoms, vitamins, coenzymes, mineral substances and antioxidants or other active ingredients useful in the indications coming within the framework of the invention described herein.

Suitably, the distribution of the composition to the various users is done by means of containers containing:

L-carnitine or alkanoyl L-carnitine acid fumarate, or

a composition containing L-carnitine or alkanoyl L-carnitine acid fumarate as active ingredient, or

L-carnitine or alkanoyl L-carnitine inner salt or one of its pharmaceutically acceptable salts and fumaric acid in a mixture with one another or packaged separately in substantially equimolar amounts, the container bearing an indication (e.g. a label) that the L-carnitine or alkanoyl L-carnitine acid fumarate or the composition containing either the mixture of L-carnitine or alkanoyl L-carnitine and fumaric acid or said ingredients packaged separately are suitable for reducing the risk of onset of organ ischemia, and for preventing and/or therapeutically treating it, particularly as affecting the cardiocirculatory apparatus, in a broad range of users or patients.

The dosage and posology will be determined by the primary care physician according to the extent of the disease to be treated and the patient's general condition.

It has also been found that it is possible to administer 1-6 g/day, and preferably 2-4 g/day, of L-carnitine, or an equivalent amount of alkanoyl L-carnitine acid fumarate.

In a first preferred embodiment of the invention, L-carnitine acid fumarate (hereinafter referred to for the sake of brevity as carnitine fumarate) is used. Preferred examples of the alkanoyl are acetyl and propionyl; butyryl and isovaleryl are also preferred.

The following examples further illustrate the invention.

### EXAMPLE 1

Effect of the Administration of L-carnitine Fumarate on the Perfused Heart

In this example, the low-pressure or low-flow ischemia model was used, which is a model recognised as valid for cardiac ischemia (Bolukoglu, H. et al. Am. J. Physiol. 1996: 270; H817-26).

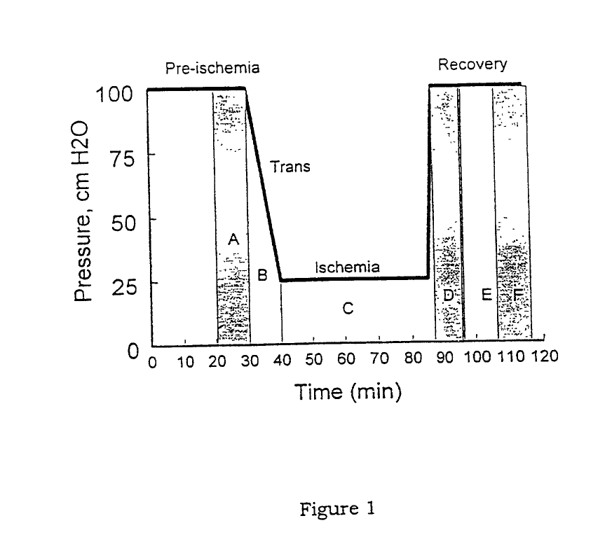

The treatment schedule is illustrated in FIG. 1, in which the letters A-F denote the heart effluent sampling times for the measurement of metabolites. The hearts are removed from the animals and mounted on a Langerdorff appliance. The perfusion medium replacing the blood was a Krebs-Heinsleit standard bicarbonate buffer containing glucose 12 mM as energy source for cardiac metabolism.

After 30 minute perfusion at a pressure of 100 cm of water, ischemia was induced by reducing the perfusion pressure of the heart to 25 cm of water, thus reducing coronary flow from approximately 2 ml/min to approximately 0.3 ml/min. Reduction of the perfusion pressure gives rise to ischemia, since the heart will pump the fluid in the low-perfusion area rather than via the coronary bloodstream, supplying the flow to the heart.

This control model was compared with hearts perfused with L-carnitine 10 mM or L-carnitine fumarate 10 mM.

Cardiac function was tested in three different ways.

In the first, the NRM ³¹P signal was monitored in real time.

This signal provides the best indication of the energy status of the heart.

In the second, the haemodynamics of the heart was measured by means of a pressure transducer mounted to measure the perfusion pressure. The haemodynamic measurements include heart rate, relative dP/dt (measurement of the contraction force of the heart) and the cardiac contraction amplitude. Coronary flow was also measured as an indicator of the heart's ability to provide oxygen and energy for its own metabolism.

In the third type of test, the metabolites and the enzyme LDH released by the heart were analysed in the effluent. The release of LDH indicates damage to cardiac tissue. The release of metabolites by the heart was tested by means of mass spectrometry coupled with gas chromatography.

The results of the experiments show that the hearts treated with carnitine fumarate have reduced release of LDH; the reserves of high-energy phosphate after 45 minutes of ischemia are greater in treated hearts, as indicated by the increase in creatine phosphate observed at NMR and the profile of the metabolites released indicates that the treated heart generates less lactate, but more malate. A high lactate level indicates intense anaerobic metabolism and acidosis. The increase in malate indicates that fumarate is metabolised by the heart to yield a system of intermediates of the citric acid cycle favourable to the heart. Haemodynamic function, as indicated by the postischemic cardiac contraction amplitude and by coronary flow, is greater in hearts treated with carnitine fumarate.

### EXAMPLE 2

The procedures of example 1 were substantially repeated, with the addition of a treatment with carnitine alone as a further control.

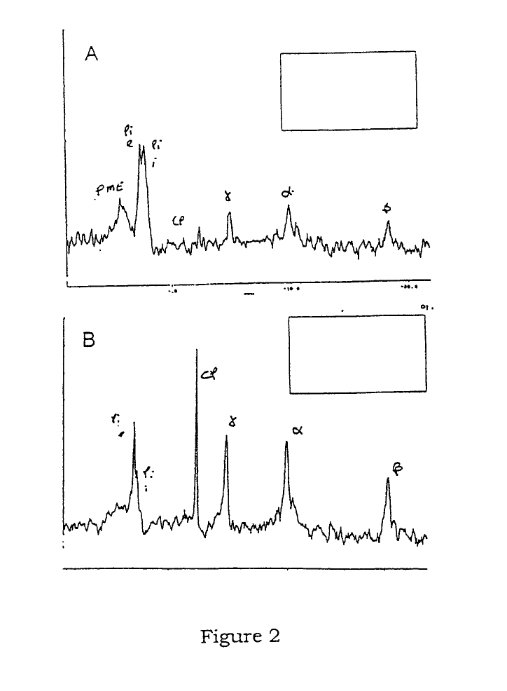

The results are given in FIGS. 2-6, where:

FIG. 2 illustrates the effect of carnitine (A) and carnitine fumarate (B) on creatine phosphate and ATP. The data were evaluated after 40 minutes of ischemia. CP indicates creatine phosphate and α, β and γ denote the phosphate peaks of ATP; as can be seen in part (A) of the figure, the ATP peaks are lacking in the absence of fumarate.

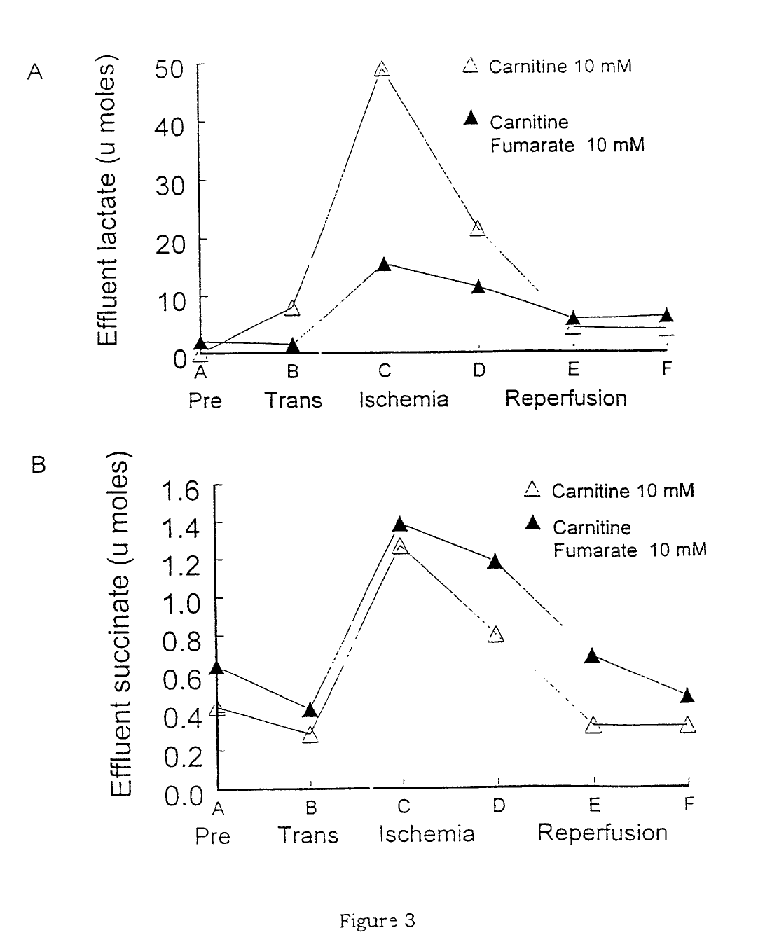

FIG. 3 shows the comparison between lactate (A) and succinate (B) released by the heart, as measured in the effluent. The lactate reduction indicates the favourable effect of carnitine fumarate. The low amount of succinate as compared to lactate indicates that the generation of ATP as a result of the reduction of fumarate to succinate is not the main source of anaerobic ATP.

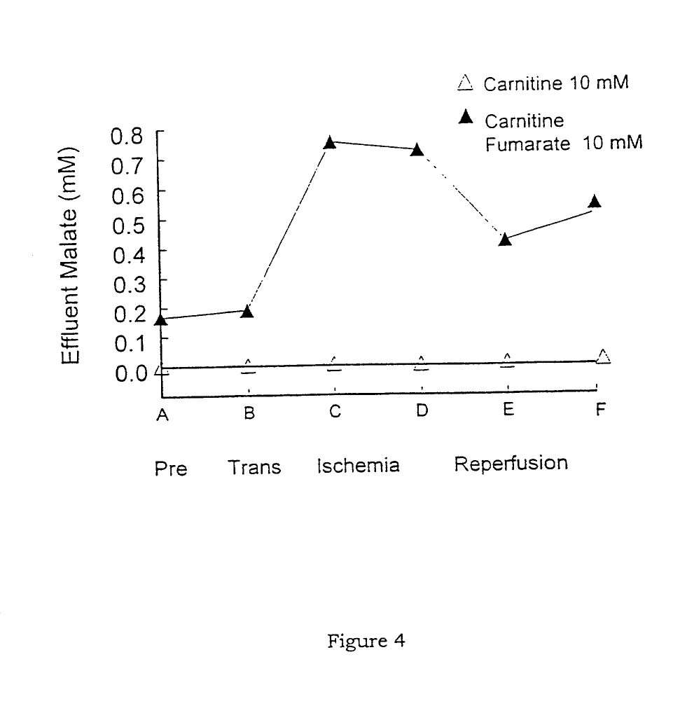

FIG. 4 illustrates the release of malate. The greater malate levels in the treated heart indicate that fumarate enters the cardiac mitochondrion and is metabolised in the TCA cycle.

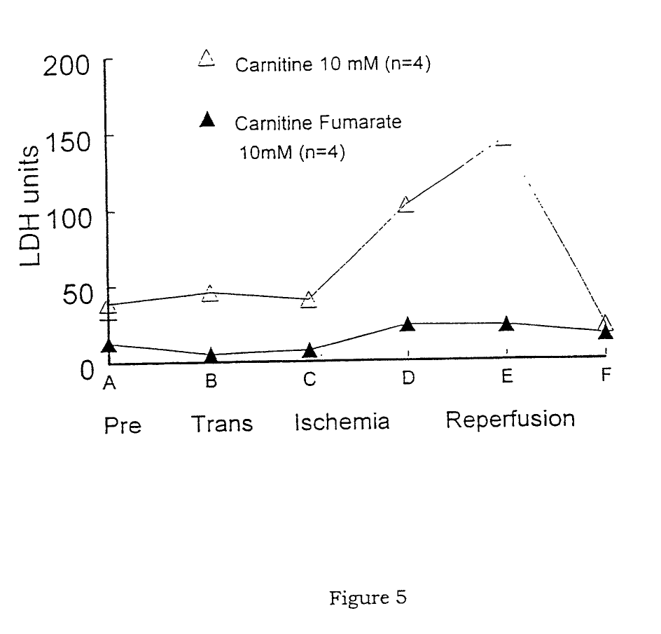

FIG. 5 illustrates the release of LDH. The greater LDH levels in controls indicate that carnitine fumarate affords protection against ischemic damage.

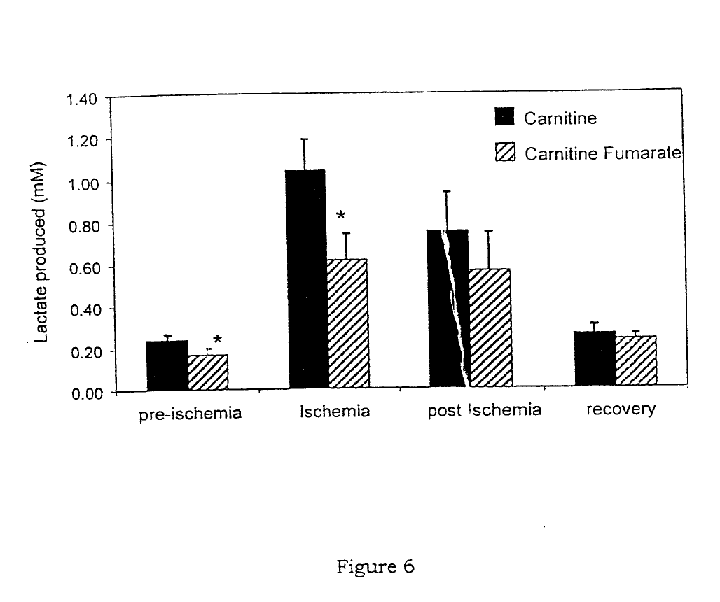

FIG. 6 illustrates lactate production.

1. Use of L-carnitine or alkanoyl L-carnitine acid fumarate or of one of its alkanoyl derivatives with formula 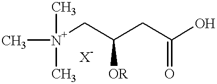
where R is hydrogen or an alkanoyl group with from 2 to 8 carbon atoms and X⁻ is the acid fumarate anion, for the preparation of a composition suitable for reducing the risk of onset of organ ischemia, and for preventing and/or therapeutically treating it, particularly as affecting the cardiocirculatory apparatus, with the proviso that said composition does not contain antioxidants.

2. Use according to claim 1, in which said composition is useful in cardiac ischemia.

3. Use according to claim 1, in which said composition is useful in renal ischemia.

4. Use according to any of claims 1-4, in which R is hydrogen.

5. Use according to any of claims 1-4, in which R is acetyl.

6. Use according to claim 1, in which R is propionyl.

7. Use according to any one of claims 1-6, where said composition takes the form of a medicine.

8. Use according to any one of claims 1-6, in which said composition can be administered orally, in the form of a food supplement.

9. Use according to claim 7, in which the composition can be administered orally or parenterally.

10. Use according to any one of claims 1-9, in which the composition takes the form of tablets, pills, granules, syrups, ampoules or drops.

11. Use according to any one of claims 1-10, in which the composition which can be administered orally or parenterally is suitable for the administration of 1-6 g/day, preferably 2-4 g/day, of L-carnitine acid fumarate or an equivalent amount of alkanoyl L-carnitine acid fumarate.

12. Container containing:
a) L-carnitine acid fumarate; or
a composition containing L-carnitine acid fumarate or alkanoyl L-carnitine acid fumarate as active ingredient, or L-carnitine or alkanoyl L-carnitine inner salt or one of its pharmacologically acceptable salts and fumaric acid in a mixture with one another or packaged separately, in substantially equimolar amounts, the container bearing an indication that the L-carnitine acid fumarate or the composition containing it or the mixture of L-carnitine and fumaric acid or these ingredients packaged separately are suitable for reducing the risk of the onset of organ ischemia, and for preventing and/or therapeutically treating it, particularly as affecting the cardiocirculatory apparatus.

13. Container according to claim 12, in which the composition contains at least one additional active ingredient and/or pharmacologically acceptable excipient.

14. Container according to claim 13, in which the additional active ingredient is selected from the group consisting of alkanoyl L-carnitines in which the alkanoyl has 2-6 carbon atoms, vitamins, coenzymes, mineral substances and antioxidants.
